(19) United States
(12) Patent Application Publication
(10) Pub. No.: US 2011/0288637 A1
(43) Pub. Date: Nov. 24, 2011

(54) Percutaneous Mitral Annular Stitch to Decrease Mitral Regurgitation

(76) Inventor: Eduardo De Marchena, Miami, FL (US)

(21) Appl. No.: 13/141,498

(22) PCT Filed: Feb. 11, 2010
(86) PCT No.: PCT/US2010/023968

§ 371 Date: Jun. 29, 2011

Related U.S. Application Data
(60) Provisional application No. 61/151,661, filed on Feb. 11, 2009.

(51) Int. Cl. A61F 2/24

(52) U.S. Cl. 623/2.11; 623/2.36

(57) ABSTRACT

This invention relates to methods and devices for treatment of regurgitation by connecting the anterior and posterior mitral annulus with a plurality of stitches or anchors called “mitral annulus bands”, instead of anchoring via the coronary sinus.

### CROSS REFERENCE TO RELATED APPLICATIONS

This application claims priority benefit under 35 USC 119(e) to U.S. Ser. No. 61/151,661, entitled Percutaneous Mitral Annular Stitch to Decrease Mitral Regurgitation, the contents of which are incorporated herein in their entirety.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO A SEQUENCE LISTING

Not applicable.

### BACKGROUND

1. Field of the Invention

This invention relates to a catheter system for attaching a mitral annulus band to posterior and anterior portions of a mitral annulus or the posterior and anterior valve itself of a mitral valve of a heart of a patient and to such a mitral annular band that can be attached with the catheter system. The mitral annulus band s structured to decrease a distance between the posterior and anterior portions of the mitral annulus while in operation to alleviate mitral regurgitation in the heart of the patient. The device may be attached to the annulus of the valve itself or the valve tissue.

### BACKGROUND OF THE INVENTION

Mitral regurgitation (MR) is characterized by the improper closure of the mitral valve which results in blood flowing backward into the left atrium. In the functional type of mitral regurgitation (FMR)), regurgitation is a byproduct of a primary disease process within the left ventricle—e.g. left ventricular enlargement. There are no medications to treat FMR, but there are medications that may be used to treat its symptoms. For patients with moderate to severe FMR, surgery is recommended, and the most common surgical approach introduces a full or partial collar-like structure around the mitral valve's base to reduce the circumference of the mitral annulus. In the past, open heart surgery was necessary to perform this remodeling of the annulus; however, percutaneous techniques are now being developed to avoid the need for open heart surgery. The most common conventional percutaneous approaches for the correction of FMR involve using various devices to remodel the posterior annulus to cause distraction in the septal direction. These devices are delivered through and/or anchored in the coronary sinus and annulus at different locations in a patient's heart, the ultimate success of these procedures varies widely.

U.S. published patent application 20070112424 discloses techniques for treating mitral valve insufficiencies such as mitral valve leakage due to prolapse, papillary muscle dysfunction, or annular dilation, including systems and methods for treating a leaking mitral valve in a minimally invasive manner and further pertain more generally to magnetic guidance and/or fastener delivery systems used for approximating or otherwise operating on tissue.

U.S. Pat. No. 6,402,781 discloses methods comprising the steps of transvenously advancing a prosthesis into the coronary sinus, and deploying at least a portion of the prosthesis within the coronary sinus to reduce the diameter of the mitral annulus.

U.S. published patent application 20060058817 discloses devices, systems and methods for enhanced treatment of a cardiac valve annulus such as a mitral valve annulus, including methods of contacting an anchor delivery device with a length of a valve annulus, delivering a plurality of coupled anchors from the anchor delivery device to secure the anchors to the annulus, and drawing the anchors together to circumferentially tighten the annulus.

U.S. published patent application 20080140191 discloses a device affects the mitral valve annulus geometry of a heart that includes a first anchor configured to be positioned within and anchored to the coronary sinus of the heart adjacent the mitral valve annulus within the heart and a second anchor configured to be positioned within the coronary sinus of the heart proximal to the first anchor and adjacent the mitral valve annulus within the heart.

U.S. published patent application discloses an implant sized and configured to be positioned in a left atrium above the plane of a native mitral heart valve annulus having leaflets that, when deployed, engages a wall of the left atrium above the plane of the native mitral valve annulus to interact with movement of the leaflets of the mitral heart valve to affect mitral heart valve function. The implant is deployed into the left atrium through an intravascular access path that extends from a right atrium through a septum and into a left atrium.

PCT published patent application discloses a valve prostheses for secure, aligned placement to a heart annulus, placed with transcatheter techniques, the prostheses includes resilient ring, plural leaflet membranes mounted with respect to the ring, plural positioning elements movably mounted with respect to the ring, wherein each positioning element defines proximal, intermediate, distal tissue engaging regions configured and dimensioned to engage separate corresponding areas of anatomical structure, including first, second, third elongate tissue-piercing elements to stabilize position of the valve with respect to anatomical structure.

There is thus a need for improved percutaneous systems and techniques for the correction of FMR.

### BRIEF SUMMARY OF THE INVENTION

A new approach for percutaneous FMR treatment is provided according to preferred embodiments of the present invention. To avoid the problem of conventional approaches, the current system can be used to connect the anterior and posterior annulus with a plurality of stitches or anchors called “mitral annulus bands”, instead of anchoring via the coronary sinus. Additionally, the current system can anchor via the tissue of the mitral valve itself, by anchoring through the valve, near the annulus. In one preferred embodiment, a catheter system is provided to deliver one or more bands to the atrial side of the mitral annulus across the mitral inflow plan to remodel the mitral annulus of the patient's heart. In another preferred embodiment, a catheter system is provided to directly anchor one side of the mitral annulus band in the posterior annulus and the other side of the mitral annulus band in the anterior annulus or the corresponding part of the valve tissue itself. The catheter system can include at least one catheter, at least one deployment, at least one tightening and/or trimming device that can be used through a lumen of the catheter, and at least one mitral annulus band. The mitral annulus band can include a first clip structured to attach to a posterior portion of the mitral annulus and a second clip structured to attach to an anterior portion of the mitral annulus of a patient's heart. The mitral annulus band can also include a cross member between the first and second clips in which a length of the cross member can be adjustable when the first and second clips are attached to thereby decrease a distance between the posterior and anterior portions of the mitral annulus to decrease an amount of mitral regurgitation, i.e. the valve leafs can be moved closer together to allow them to close more fully.

### BRIEF DESCRIPTION OF THE DRAWINGS

FIG. 1 is a graphic representation of a heart in cross-section and shows a normal left ventricle.

FIG. 2 is a graphic representation of a heart in cross-section and shows a normal left ventricle an enlarged left ventricle and mis-aligned mitral valve.

FIG. 3 is a graphic representation of a heart in cross -section and shows a trans-septal puncture to allow the catheter to access the left atrium.

FIG. 4 is a graphic representation of a heart in cross-section and shows catheter deployment of a clip with suture to the posterior mitral annulus.

FIG. 5 is a graphic representation of a heart in cross-section and shows catheter deployment of a second clip with suture to the anterior mitral annulus.

FIG. 6 is a graphic representation of a heart in cross-section and shows sutured clips in place and being tightened to bring together the posterior and anterior annulus.

FIG. 7 is a graphic representation of a heart in cross-section and shows sutured clips in place and the posterior and anterior annulus drawn to proper dimension.

FIG. 8 is a graphic representation of a mitral valve.

FIG. 9 is a graphic representation of a mitral valve having two bands deployed.

### DETAILED DESCRIPTION OF THE INVENTION

A dilated mitral annulus in dilated cardiomyopathy, the mitral valve fails to close completely, allowing regurgitation of blood during systole. The present inventive subject matter addresses this problem by realigning the mitral valve without the complications of anchoring the anteromedial and posterolateral leaflets. Using a steerable catheter that is deployed to the left atrium, a clip is advanced for delivery to the mitral annulus. By advancing the catheter, it pushes each clip into place for implantation into the mitral annulus. When a first clip is attached to the posterior mitral annulus and a second clip is attached to the anterior mitral annulus, a suture is drawn between them to provide a tether which draws the leaflets back into proper alignment, thus eliminating or reducing pathological mitral regurgitation. Prolapsed and/or non-coapting mitral valves can be remodelled using the simple method of the present invention.

The terms clip or anchor are functionally synonymous and describe devices for attaching to the mitral annulus.

The terms suture or tether or band refer to the length of material that spans the mitral valve and provides the longitudinal and/or lateral force to remodel the mitral valve from a pathological state to an improved geometry.

In a preferred embodiment, the material used for the suture is nitinol. Although it is contemplated that any suitable biocompatible material is within the scope of the invention. For attachment, it is contemplated that surgical clips, surgical anchors, and/or other known surgical attachment devices and techniques are within the scope of the present invention. In a preferred embodiment, multiple sutures or tethers, are used. The present inventive subject matter contemplates from 1 to 5 deployed suture-clip combinations can be installed. The intent is to reduce the annular diameter of the mitral valve in order to address the mitral regurgitation.

The bands are deployed, in a preferred non-limiting embodiment across the mitral inflow plane, although other useful geometries are also contemplated as within the scope of the present invention.

The delivery of the catheter to the correct location uses transesophageal echocardiography, intra-cardiac echocardiography, and fluoroscopy. Verification of the efficacy of the procedure is performed by echo Doppler and LV angiography.

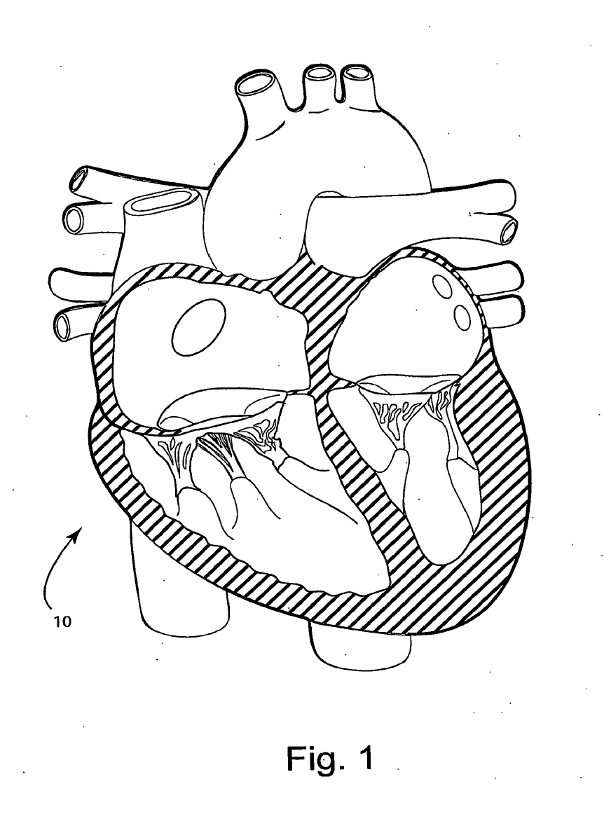

Referring now to the FIGURES, FIG. 1 shows normal heart 10 in cross-section and shows a normal left ventricle with a properly aligned mitral valve.

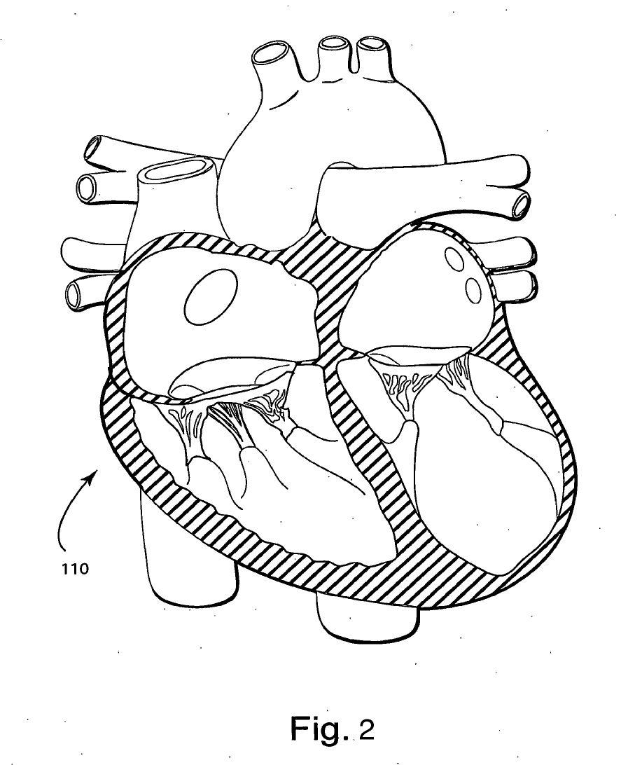

FIG. 2 shows abnormal heart 110 with an enlarged left ventricle and mis-aligned mitral valve.

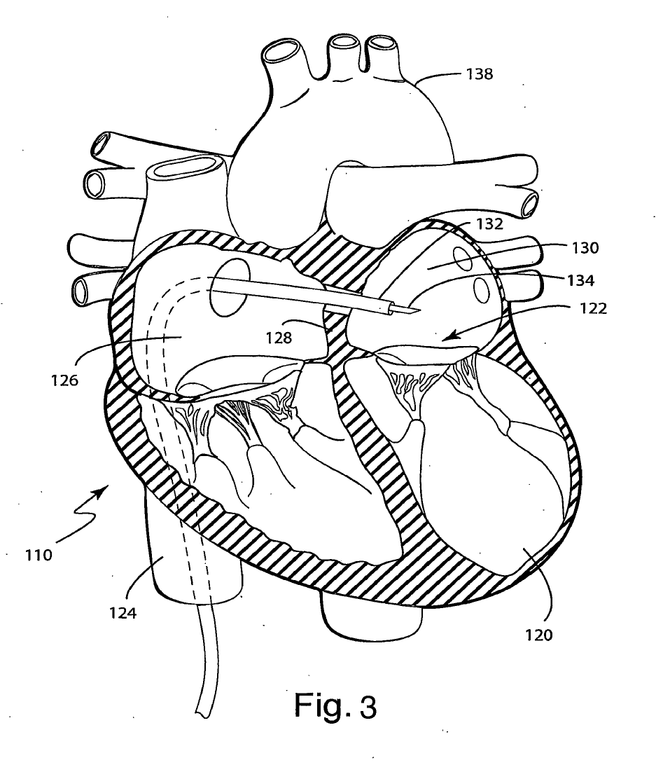

FIG. 3 shows step 1 of a preferred method of the present invention where a trans-septal puncture 128 is performed to allow the catheter 132 to access the left atrium 130. Cutting tip 134 is withdrawn once access to the left atrium 130. FIG. 3 shows mitral valve 122 and enlarged left ventricle 120.

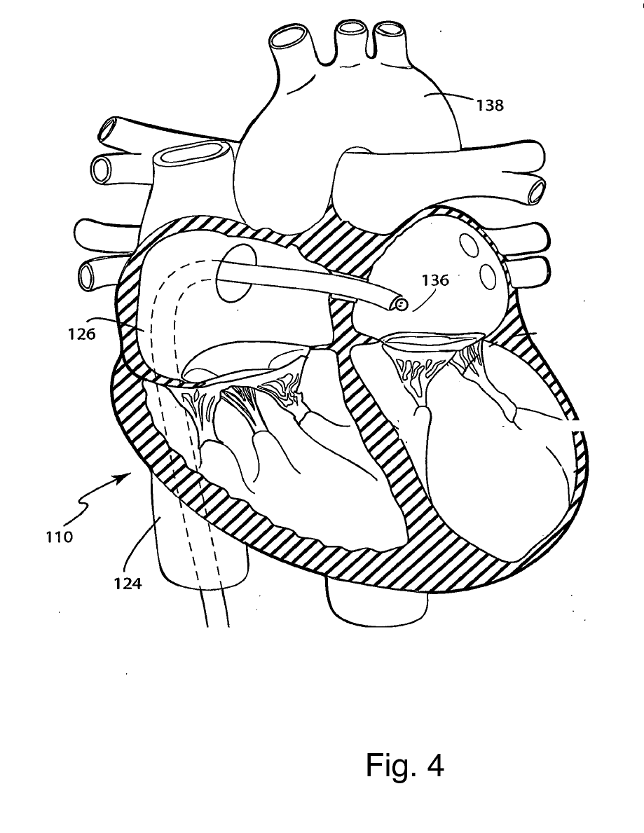

FIG. 4 shows step 2 of a preferred method of the present invention where catheter deployment of a clip with suture 136 is performed and the clip with suture 136 is attached to the posterior mitral annulus.

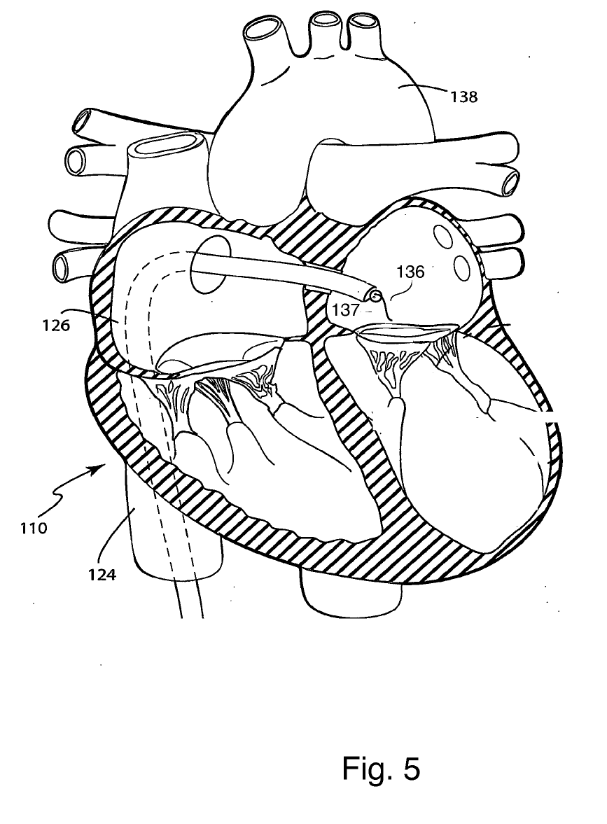

FIG. 5 shows step 3 of a preferred method of the present invention where catheter deployment of a second clip with suture 137 is performed and the clip with suture 137 is attached to the anterior mitral annulus.

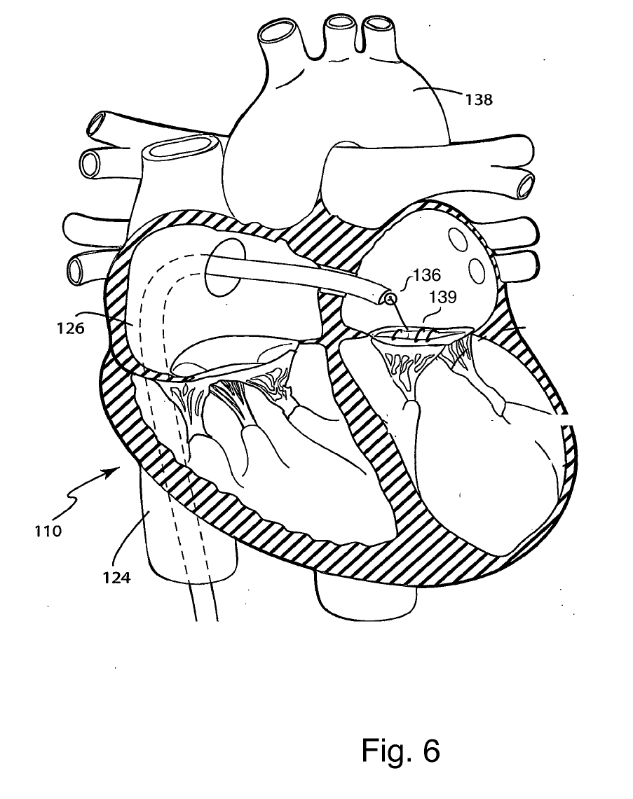

FIG. 6 shows step 4 of a preferred method of the present invention where sutured clips are in place and step 5 where the clips with sutures are being tightened to bring together the posterior and anterior annulus into proper alignment.

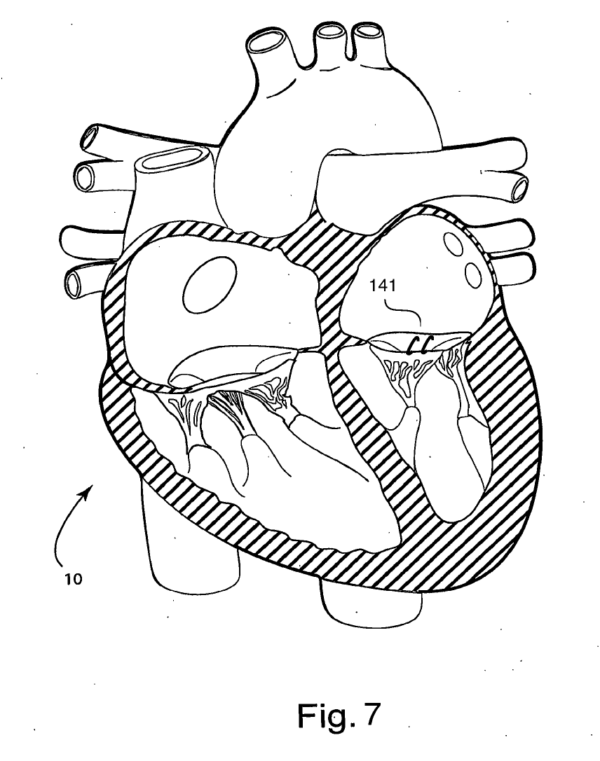

FIG. 7 shows step 6 of a preferred method of the present invention where sutured clips in place and the posterior and anterior annulus drawn to proper dimension and the catheter has been withdrawn from the left atrium.

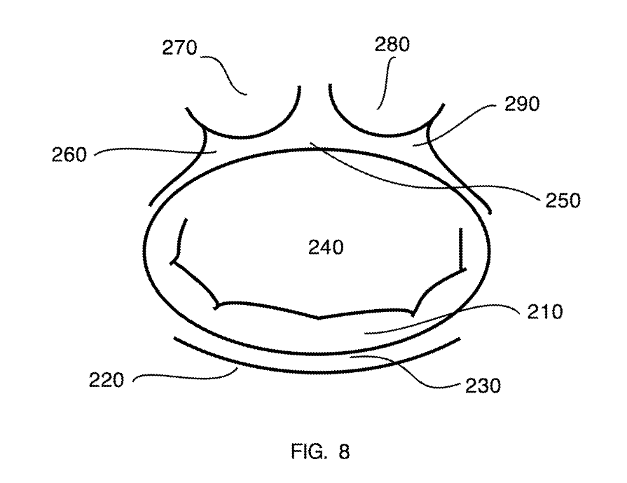

FIG. 8 shows a mitral valve having posterior leaflet 210, coronary sinus 220 and posterior annulus 230. Anterior leaflet 240 is shown having anterior annulus 250, left fibrous trigone 260, left coronary sinus 270, non-coronary sinus 280, and right fibrous trigone 290.

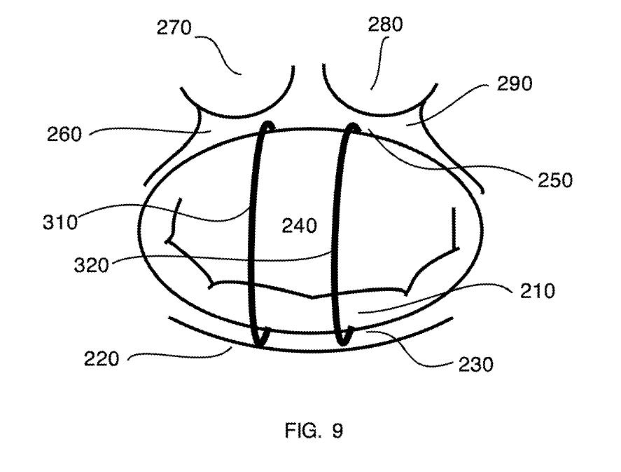

FIG. 9 shows a mitral valve having stitch/suture/tether 310 and stitch/suture/tether 320 deployed from posterior annulus 230 to anterior annulus 250, and spanning the mitral valve.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed:

1. A catheter system, comprising:
a catheter defining a lumen therethrough;
a band deployment unit that is structured to deploy a mitral annulus band through said lumen of said catheter; and
a mitral annulus band adapted to be deployed through said lumen of said catheter by said band deployment unit,
wherein said mitral annulus band is structured to attach to a posterior portion of a mitral annulus or posterior portion of mitral valve tissue of a mitral valve of a heart of a patient and said mitral annulus band is structured to also attach to an anterior portion of said mitral annulus or anterior portion of mitral valve tissue of said mitral valve, and
wherein said mitral annulus band is structured to decrease a distance between said posterior and anterior portions of said mitral annulus while in operation to alleviate mitral regurgitation in said heart of said patient.

2. The catheter system according to claim 1, further comprising wherein said mitral annulus band comprises a first clip structured to attach to said posterior portion of said mitral annulus and a second clip structured to attach to said anterior portion of said mitral annulus.

3. The catheter system according to claim 2, further comprising wherein said mitral annulus band comprises a cross member between said first clip and said second clip,
wherein a length of said cross member is adjustable when said first clip is attached to said posterior portion of said mitral annulus and when said second clip is attached to said anterior portion of said mitral annulus to thereby decrease said distance between said posterior and anterior portions of said mitral annulus to decrease an amount of mitral regurgitation.

4. The catheter system according to claim 3, further comprising wherein said cross member of said mitral annulus band comprises suture.

5. The catheter system according to claim 3, further comprising wherein said band deployment unit is also structured to adjust said length of said cross member of said mitral annulus band.

6. The catheter system according to claim 3, further comprising a band tightening unit that is structured to adjust said length of said cross member of said mitral annulus band through said catheter.

7. The catheter system according to claim 3, further comprising wherein said band deployment unit is also structured to trim portions of said cross member of said mitral annulus band.

8. The catheter system according to claim 3, further comprising a band trimming unit that is structured to trim portions of said cross member of said mitral annulus band through said catheter.

9. A mitral annulus band, comprising:
a first clip structured to attach to a posterior portion of a mitral annulus of a patient's heart;
second clip structured to attach to an anterior portion of said mitral annulus of said patient's heart; and
a cross member between said first clip and said second clip,
wherein a length of said cross member is adjustable when said first clip is attached to said posterior portion of said mitral annulus and when said second clip is attached to said anterior portion of sad mitral annulus to thereby decrease a distance between said posterior and anterior portions of said mitral annulus to decrease an amount of mitral regurgitation.
